(19) United States
(12) Patent Application Publication
(10) Pub. No.: US 2021/0169534 A1
(43) Pub. Date: Jun. 10, 2021

(54) Apparatus for Spinal Surgery of Inserting Rod

(71) Applicant: Solco Biomedical Co., Ltd., Pyeongtaek-si (KR)

(72) Inventors: Mi-rim HYEON, Hwaseong-si Gyeonggi-do (KR); Il KIM, Gangnam-gu Seoul (KR); Hwi Geun YU, Pyeongtaek-si Gyeonggi-do (KR)

(73) Assignee: Solco Biomedical Co., Ltd., Pyeongtaek-si (KR)

(21) Appl. No.: 16/804,682

(22) Filed: Feb. 28, 2020

(30) Foreign Application Priority Data
Dec. 9, 2019 (KR) 10-2019-0162674

(51) Int. Cl. A61B 17/70

(52) U.S. Cl. CPC A61B 17/7085; A61B 17/7089

(57) ABSTRACT

Provided is a spinal surgery apparatus for inserting a rod, which allows a rod forming an implant structure to be easily inserted and fixed, thus facilitating spinal surgery and minimizing invasions during the spinal surgery, thus helping a patient's recovery.

The spinal surgery apparatus for inserting a rod includes a handle gripped by a user, a fixing shaft connected to the handle, having on a lower end thereof a fixing part to which the rod is fixed, and separating the fixing part from the rod while the fixing shaft is tilted forwards, an actuating part including a connecting shaft inserted into the handle, and an actuating member installed on an end of the connecting shaft to move the connecting shaft up and down by rotation, and a rod push shaft inserted into the fixing shaft, and simultaneously coming at an upper end thereof into contact with a lower end of the connecting shaft, so that the rod push shaft is moved down by an operation of the actuating part, thus pushing and fixing the rod.

### BACKGROUND OF THE DISCLOSURE

### Field of the Invention

The present disclosure relates to a spinal surgery apparatus for inserting a rod, configured to insert the rod into an implant structure.

### Related Art

Generally, the spine is composed of 24 bones (excluding sacral spine), which are connected by joints called disks between the respective bones to support the spine and absorb shocks. Thereby, the spine can help to maintain the posture of the human, form the basis of movement, and play an important role in protecting internal organs.

However, if an abnormal posture is maintained for a long time, the spine suffers from degenerative diseases due to aging or is subjected to external shocks, the disk of the spinal joint may be damaged, thus resulting in spinal disk diseases. These spinal disk diseases compress nerves connected to respective parts of the human body via the spinal joints, thus causing pain.

Therefore, patients having the spinal disk diseases undergo the following procedure: a disk of a damaged region is removed so that a damaged part of the vertebra is not pressed or compressed, an artificial aid (cage) made of hollow metal or plastic materials is filled with bone chips to be inserted into the region from which the disk has been removed, and then a screw is fixedly inserted into the vertebra of each of upper and lower regions of the damaged disk. Thereafter, a rod is connected to the screw to secure a distance between the vertebrae and thereby allow bone fusion to be normally performed.

The spinal surgery may be performed by incising the skin of a damaged spinal region, removing or leaving the damaged disk, fixedly inserting the screw into the vertebra of each of upper and lower portions of the damaged disk, connecting the rod thereto, and fastening a bolt to the screw of the fixed disk.

That is, an implant structure placed in the vertebra may be built by fixedly inserting the rod into the screw that is fixed using the spinal surgery apparatus.

The spinal surgery apparatus is inserted into the spinal region with the rod being fixed to an end of the apparatus, and is fixedly inserted into an upper portion of the screw to connect a previously inserted screw to the screw. The spinal surgery is advantageous to patients when it is minimally invasive. However, in the state where the spinal surgery apparatus moves backwards after the rod is fixed, the apparatus is taken out from the spinal region. Thus, in order to secure a space, a large incision is made around the spinal region, thereby increasing an invasion.

As such, if the invasion increases during the spinal surgery, the bleeding of a surgical site is increased, the recovery is delayed, and the scar of the surgical site is also increased.

### CITED REFERENCE

### Patent Document

(Patent Document 1) Korean Patent No. 10-1111666 (Title of Invention: Apparatus of spinal surgical operation for minimally invasive surgery, laid open on Aug. 30, 2010)

(Patent Document 2) Korean Patent Publication No. 10-2019-0108269 (Title of Invention: Spinal surgery device, laid open on Sep. 24, 2019)

### SUMMARY OF THE INVENTION

The present disclosure provides a spinal surgery apparatus for inserting a rod, which allows a rod forming an implant structure to be easily inserted and fixed, thus facilitating spinal surgery and minimizing invasions during the spinal surgery, thus helping a patient's recovery.

In an aspect, a spinal surgery apparatus for inserting a rod may include a handle gripped by a user, a fixing shaft connected to the handle, having on a lower end thereof a fixing part to which the rod is fixed, and separating the fixing part from the rod while the fixing shaft is tilted forwards, an actuating part including a connecting shaft inserted into the handle, and an actuating member installed on an end of the connecting shaft to move the connecting shaft up and down by rotation, and a rod push shaft inserted into the fixing shaft, and simultaneously coming at an upper end thereof into contact with a lower end of the connecting shaft, so that the rod push shaft is moved down by an operation of the actuating part, thus pushing and fixing the rod.

The fixing part may include a fixing hole into which an end of the rod is fixedly inserted, and a fixing groove formed above the fixing hole with a step therebetween, thus allowing the fixing shaft to be tilted.

The fixing groove may define a moving space of the fixing shaft, and inclination parts may be formed on both sides of a lower portion of the fixing shaft.

A tilting angle of the fixing shaft may range from 30° to 60° with respect to a vertical state of the fixing shaft.

An inflow groove may be longitudinally formed in a rear portion of the fixing shaft to introduce blood therein.

A spinal surgery apparatus for inserting a rod according to the present disclosure is advantageous in that it allows a rod forming an implant structure to be easily inserted and fixed, thus facilitating spinal surgery and minimizing invasions during the spinal surgery, thus helping a patient's recovery and leaving only a small scar in a surgical site.

Furthermore, since it is easy to clean a spinal surgery apparatus, it is possible to effectively keep the entire apparatus clean.

### BRIEF DESCRIPTION OF THE DRAWINGS

FIG. 1 is a diagram illustrating a spinal surgery apparatus for inserting a rod in accordance with an embodiment of the present disclosure.

FIG. 2 is a diagram illustrating the configuration of the spinal surgery apparatus for inserting the rod in accordance with the embodiment of the present disclosure.

FIG. 3 is a diagram illustrating a state in which the spinal surgery apparatus for inserting the rod in accordance with the embodiment of the present disclosure is tilted.

FIG. 4 is a diagram illustrating the appearance of the spinal surgery apparatus for inserting the rod in accordance with the embodiment of the present disclosure.

FIG. 5 is a diagram illustrating a rod insertion part of the spinal surgery apparatus for inserting the rod in accordance with the embodiment of the present disclosure.

FIG. 6 is a diagram illustrating a fixing shaft of the spinal surgery apparatus for inserting the rod in accordance with the embodiment of the present disclosure.

FIG. 7 is a diagram illustrating a state in which the rod is fixedly inserted into a screw with the spinal surgery apparatus for inserting the rod in accordance with the embodiment of the present disclosure.

### DESCRIPTION OF EXEMPLARY EMBODIMENTS

Hereinafter, a spinal surgery apparatus for inserting a rod in accordance with an embodiment of the present disclosure will be described with reference to the accompanying drawings. The present disclosure is not limited or restricted by the embodiment. Further, in describing the present disclosure, specific details of known functions or configurations may be omitted to clarify the gist of the present disclosure.

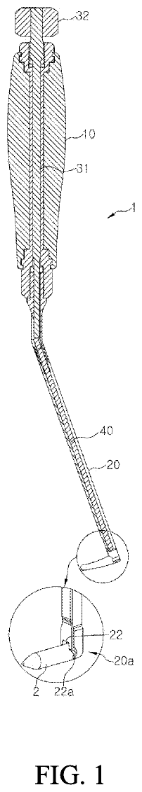

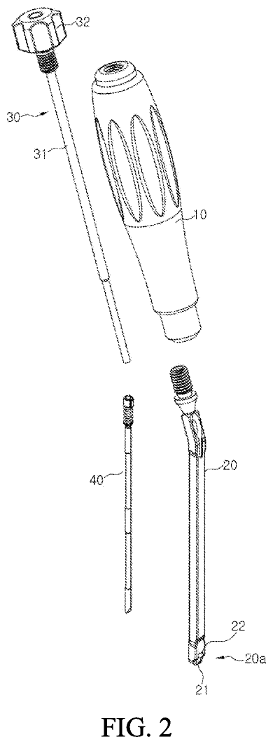

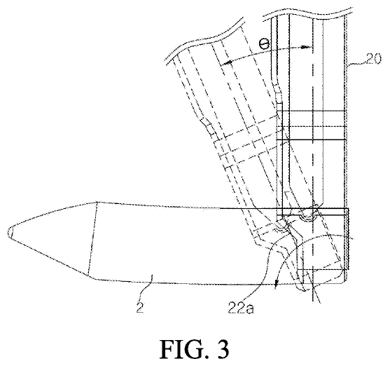

FIG. 1 is a diagram illustrating a spinal surgery apparatus for inserting a rod in accordance with an embodiment of the present disclosure. FIG. 2 is a diagram illustrating the configuration of the spinal surgery apparatus for inserting the rod in accordance with the embodiment of the present disclosure. FIG. 3 is a diagram illustrating a state in which the spinal surgery apparatus for inserting the rod in accordance with the embodiment of the present disclosure is tilted.

Referring to FIGS. 1 to 3, the spinal surgery apparatus 1 for inserting the rod in accordance with the present disclosure may include a handle 10 gripped by a user, a fixing shaft 20, an actuating part 30, and a rod push shaft 40, and may insert a rod 2 fixed to the fixing shaft 20 into a screw 3 fixed to the spine, thus building an implant structure 100.

According to this embodiment, the fixing shaft 20 of the spinal surgery apparatus 1 is connected to the handle 10, and a fixing part 20a to which the rod 2 is fixed is provided on a lower end of the fixing shaft 20. While the fixing shaft 20 is tilted forwards, the fixing part 20a may be separated from the rod 2.

The fixing shaft 20 fixedly inserts the rod 2 into the fixing part 20a provided on an end thereof. In this state, after the rod 2 is inserted into a spinal surgical site, the fixing shaft 20 is tilted forwards, so that the rod 2 is separated from the fixing part 20a. The rod 2 separated from the fixing part 20a is fixedly inserted into the screw 3 forming the implant structure 100.

Furthermore, the actuating part 30 includes a connecting shaft 31 inserted into the handle 10, and an actuating member 32 installed on an end of the connecting shaft 31 to move the connecting shaft 31 up and down by rotation. The rod push shaft 40 is inserted into the fixing shaft 20, and simultaneously its upper end comes into contact with the lower end of the connecting shaft 31, so that the rod push shaft is moved down by the operation of the actuating part 30, thus pushing and fixing the rod 2.

In other words, the spinal surgery apparatus 1 may fix or release the rod 2 by the operation of the actuating part 30 and the movement of the rod push shaft 40. If the actuating member 32 of the actuating part 30 is rotated with the rod 2 being inserted into the fixing part 20a on the lower end of the fixing shaft 20, the connecting shaft 31 moves down and the rod push shaft 40 connected therewith moves down, so that the lower end of the rod push shaft 40 may push the rod 2 and thereby the rod 2 may be fixed to the lower end of the fixing shaft 20.

According to this embodiment, the fixing part 20a forming the lower end of the fixing shaft 20 has a fixing hole 21 into which the end of the rod 2 is fixedly inserted, and a fixing groove 22 is formed above the fixing hole 21 with a step therebetween, thus allowing the fixing shaft 20 to be tilted. The fixing groove 22 may define the moving space of the fixing shaft 20, and inclination parts 22a may be formed on both sides of a lower portion of the fixing shaft.

After the rod 2 inserted into the fixing hole 21 is inserted into the screw 3, the fixing shaft 20 is tilted forwards, so that the rod 2 may be removed from the fixing hole 21. To this end, in order to tilt the fixing shaft 20, the fixing groove 22 and the inclination parts 22a are formed on the fixing part 20a.

The fixing groove 22 formed above the fixing hole 21 with the step therebetween may secure a tilting space in the lower end of the fixing shaft 20. The inclination parts 22a allow the movement of the lower end of the fixing shaft 20 to be smoothly guided.

Furthermore, the tilting angle θ of the fixing shaft 20 may range from 30° to 60° with respect to the vertical state of the fixing shaft 20. The tilting angle θ of the fixing shaft 20 is minimized to allow minimally invasive surgery during the spinal surgery. Thus, the tilting angle is preferably about 30°.

After the rod 2 is fixedly inserted into the screw 3 of the implant structure 100, the rod 2 is released by rotating the actuating member 32. In this state, the fixing shaft 20 is tilted, so that the rod 2 is removed from the fixing hole 21 of the fixing part 20a. Next, the fixing shaft 20 is lifted upwards, so that the spinal surgery apparatus 1 may be taken out from the surgical site.

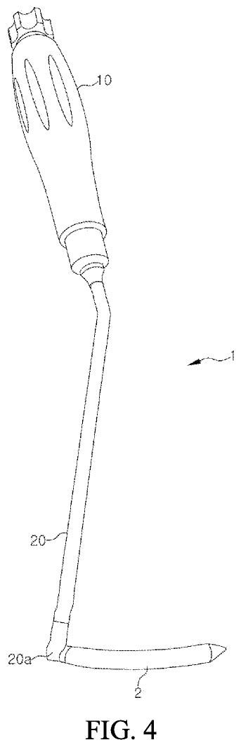

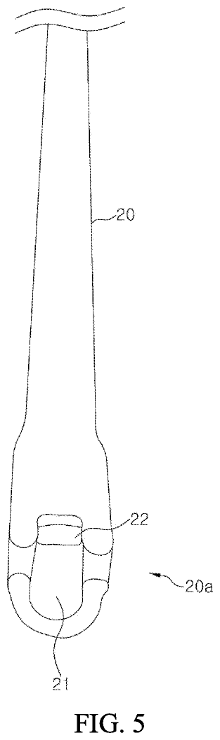

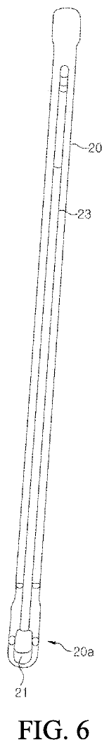

FIG. 4 is a diagram illustrating the appearance of the spinal surgery apparatus for inserting the rod in accordance with the embodiment of the present disclosure. FIG. 5 is a diagram illustrating a rod insertion part of the spinal surgery apparatus for inserting the rod in accordance with the embodiment of the present disclosure. FIG. 6 is a diagram illustrating the fixing shaft of the spinal surgery apparatus for inserting the rod in accordance with the embodiment of the present disclosure.

Referring to FIGS. 4 to 6, the spinal surgery apparatus 1 inserts the rod 2 into the spinal surgical site in the state in which the rod 2 forming the implant structure 100 is fixed to the fixing part 20a disposed on the lower end of the fixing shaft 20 connected to the handle 10. The fixed state of the rod 2 inserted into the fixing hole 21 of the fixing part 20a is released, and the fixing shaft 20 is tilted through the fixing groove 22 of the fixing part 20a, thus allowing the rod 2 to be removed from the fixing hole 21.

Furthermore, an inflow groove 23 may be longitudinally formed in a rear portion of the fixing shaft 20 to introduce blood therein. The blood introduced into the fixing shaft 20 through the inflow groove 23 can be more effectively cleaned.

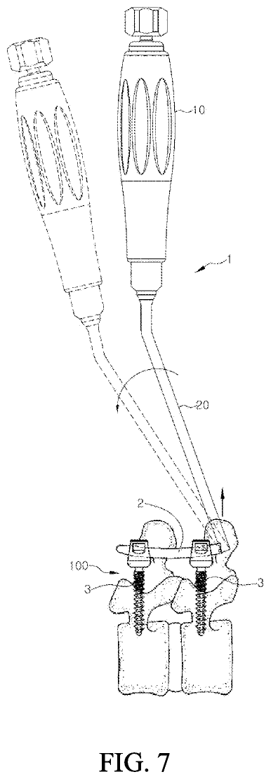

FIG. 7 is a diagram illustrating a state in which the rod is fixedly inserted into the screw with the spinal surgery apparatus for inserting the rod in accordance with the embodiment of the present disclosure.

Referring to FIG. 7, a process of fixedly inserting the rod 2 into the screw 3 of the implant structure 100 using the spinal surgery apparatus 1 will be described. First, in the state where the rod 2 is fixed to the fixing part 20a of the spinal surgery apparatus 1, the fixing shaft 20 is inserted into the surgical site and the rod 2 is inserted into the upper portion of the screw 3. In this state, the actuating member 32 of the spinal surgery apparatus 1 is rotated to release the rod 2 fixed to the fixing part 20a, and the handle 10 is tilted forwards to tilt the fixing shaft 20, thus removing the rod 2 from the fixing hole 21 of the fixing part 20a.

The fixing shaft 20 from which the rod 2 has been removed lifts the handle 10 upwards, so that the apparatus is taken out from the surgical site with the fixing shaft 20 being in a vertical position. Thus, the spinal surgery apparatus 1 allows minimally invasive surgery. In contrast, according to the related art, since the fixing shaft 20 removed from the rod 2 moves backwards and then the fixing shaft 20 is taken out from the surgical site, minimally invasive surgery is difficult.

Therefore, the spinal surgery apparatus 1 allows the rod 2 forming the implant structure 100 to be easily inserted and fixed, thus facilitating spinal surgery and minimizing invasions during the spinal surgery, thus helping a patient's recovery and leaving only a small scar in the surgical site.

Furthermore, since it is easy to clean the spinal surgery apparatus 1, it is possible to effectively keep the entire apparatus clean.

Although the present disclosure has been shown and described with reference to preferred embodiments for illustrating the principle of the present disclosure, the present disclosure is not limited to the above-described configuration and operation. Rather, those skilled in the art will appreciate that many changes and modifications of the present disclosure may be made without departing from the spirit and scope of the appended claims.

What is claimed is:

1. A spinal surgery apparatus for inserting a rod, comprising:
a handle gripped by a user;
a fixing shaft connected to the handle, having on a lower end thereof a fixing part to which the rod is fixed, and separating the fixing part from the rod while the fixing shaft is tilted forwards;
an actuating part comprising a connecting shaft inserted into the handle, and an actuating member installed on an end of the connecting shaft to move the connecting shaft up and down by rotation; and
a rod push shaft inserted into the fixing shaft, and simultaneously coming at an upper end thereof into contact with a lower end of the connecting shaft, so that the rod push shaft is moved down by an operation of the actuating part, thus pushing and fixing the rod.

2. The spinal surgery apparatus of claim 1, wherein the fixing part comprises a fixing hole into which an end of the rod is fixedly inserted, and a fixing groove formed above the fixing hole with a step therebetween, thus allowing the fixing shaft to be tilted.

3. The spinal surgery apparatus of claim 2, wherein the fixing groove defines a moving space of the fixing shaft, and inclination parts are formed on both sides of a lower portion of the fixing shaft.

4. The spinal surgery apparatus of claim 1, wherein a tilting angle of the fixing shaft ranges from 30° to 60° with respect to a vertical state of the fixing shaft.

5. The spinal surgery apparatus of claim 1, wherein an inflow groove is longitudinally formed in a rear portion of the fixing shaft to introduce blood therein.
